(19) United States
(12) Patent Application Publication
(10) Pub. No.: US 2008/0004493 A1
(43) Pub. Date: Jan. 3, 2008

(54) Device for Changing the Acting Direction of an Instrument

(76) Inventor: Patrick A. Schiemann, Kehl am Rhein (DE)

Correspondence Address: GREER, BURNS & CRAIN, 300 S WACKER DR, 25TH FLOOR, CHICAGO, IL, 60606, US

(21) Appl. No.: 11/596,509

(22) PCT Filed: May 12, 2005
(86) PCT No.: PCT/EP05/05185

§ 371 Date: Jul. 31, 2007

(30) Foreign Application Priority Data
May 14, 2004 (DE) 10 2004 024 128.7

(51) Int. Cl. A61B 1/005

(52) U.S. Cl. 600/118; 606/1

(57) ABSTRACT

A device for controlling an instrument includes at least one sensor with at least one guiding zone which is guided by the user's hand during its use. Moreover, the claimed device includes a spacer whose first end is rigidly fixed to the sensor. At least one controller which carries at least one instrument to be controlled is arranged at a second end of the spacer. The sensor senses a change in position of the guiding zone, away from a neutral position, and the controller controls the acting direction of the instrument on the basis of the data acquired by the sensor.

The present invention relates to a device for changing the acting direction of an instrument. In particular the present invention relates to a device for changing the acting direction of an endoscopic instrument such as it is used in endoscopic surgery, such as laparoscopic or arthroscopic operations, or in the field of cardiology, such as in treatment of coronary artery diseases or in heart valve surgery.

Although the invention will be described below by way of example for an endoscopic instrument or tool, the invention is not limited to this field of application but it is understood to be a possibility usable in multiple technical areas of controlling tools or instruments or their acting directions in places that are not readily accessible.

Many known devices for controlling instruments from the prior art comprise mechanisms of complicated designs to control said instrument at the operating site. Due to the complexity of said mechanisms, most known devices for controlling instruments are very expensive in manufacture and for one-time use only since it is highly expensive to achieve a satisfactory cleaning and sterilising of said complex mechanisms after use, as is required for re-use in surgery.

Other known devices for controlling instruments also show the disadvantage that the means with which the operator controls said instrument is not configured so as to allow to quickly put down an instrument and take up another e.g. fulfilling a different function while maintaining sterile conditions, without impeding or limiting the functionality of the device.

It is therefore the object of the present invention to provide a device for controlling instruments which overcomes the disadvantages of the known devices and in particular offers the advantage over the prior art of a simplified structure and low manufacturing costs while maintaining substantially unimpeded functionality.

This object is fulfilled according to the invention by a device having the features of claim 1. Further embodiments of the present invention are the objects of the subclaims.

Thus, the device according to the invention comprises at least one sensor means comprising at least one guiding zone which the user guides by hand when using the device for controlling an instrument. The device according to the invention furthermore comprises a spacer rigidly fixed to said sensor means in the region of a first end, and a controller carrying at least one instrument or tool to be controlled, and which is arranged at a second end of said spacer.

According to the invention the sensor means comprises means which detect changes in position of the guiding zone out of a neutral position or which allow to determine the position of the guiding zone relative to the neutral position. Furthermore the controller comprises means which control the acting direction of the instrument arranged at the controller on the basis of data acquired by the sensor means about the position or change in position of the guiding zone relative to the neutral position.

The spacer in the device according to the invention is preferably an integral elongated component, particularly preferably comprising one or more hollow spaces, preferably tubular hollow spaces or lumina. It is particularly preferred that the spacer is a rigid component; however the device according to the invention also includes embodiments comprising a spacer that is flexible at least in some areas. In addition, embodiments of the present invention are conceivable where the spacer consists of two or more components which may not be connected to one another.

The instrument to be controlled in the device according to the invention is preferably placed on a side of the controller facing away from the sensor means wherein a connection of the instrument to be controlled with the controller is established by adhesive, frictional and/or form-fit and in particular by adhesive bond, plug-in, compression, snap-on, screwed and/or bayonet connection or the like. It is in particular preferred to have a releasable connection between the controller and the instrument so as to render the device for controlling an instrument suitable for controlling a plurality of instruments which may be different in design.

In using the device according to the invention the user holds or grasps or grips, respectively, the guiding zone of the sensor means preferably in at least one hand. In another preferred embodiment the guiding zone has provided at it means which allow a suitable connection between at least one hand and in particular the fingers of the user, and the guiding zone. Such means may include depressions and/or passages into or through which the user places or threads one or more finger(s).

The device according to the invention stands out in particular in that no other connection exists between the user and the sensor means except in the area of the guiding zone which the user takes, grips and/or engages with his hand, in particular no connection that would fixate the sensor means, the spacer and/or the controller relative to the arm or another part of the body of the user.

In particular in using the device according to the invention for controlling instruments in the field of endoscopic surgery, but also in using in other technical fields, the spatial position of the device for controlling instruments is determined at least in part by a fixation point lying preferably in the area of the spacer, such as in the shape of an annular or tubular passage. Preferably, such an annular or tubular passage furthermore allows the spacer or the device according to the invention to be displaced along said passage, so as to thus allow a displacement of the fixation point along said spacer.

In a particularly preferred embodiment of the present invention the guiding zone of the sensor means is supported to be rotatable about a first axis which, in the region of connection of the spacer with the sensor means, is substantially orthogonal to the axis of the principal dimensional direction of the spacer. Thus the guiding zone is allowed to tilt about the first axis which can be determined by the means for detecting the position or changes in the position of the guiding zone of the sensor means.

It is in particular preferred that the guiding zone is furthermore supported to be rotatable about a second axis substantially orthogonal to said first axis, so as to also allow the guiding zone to tilt about said second axis, where the tilt about said second axis can also be determined by the means for detecting the position or changes in the position of the sensor means.

It is in particular preferred that said first and said second axis are positioned such that they intersect in a first intersection point which is preferably in a plane which also comprises a connecting point between the spacer and the sensor means.

Due to the rotatable support of the guiding zone about said first and second axis, said axis can be moved in a spherically or non-spherically curved plane, depending on whether or not said first and second axis intersect in said first intersection point.

Most preferably the rotatable support of the guiding zone about the first or second axis respectively is designed as a cardan joint which allows a movement of the guiding zone about the first intersection point in a spherically curved plane. The rotatable support of the guiding zone is preferably configured as a gimbal suspension relative to an axis which is substantially parallel to the axis of the principal dimensional direction of the spacer in the area of connection with the sensor means such that said axis preferably intersects with the first intersection point and the guiding zone can be tilted substantially symmetrically relative to said axis.

In a particularly preferred embodiment of the present invention the means for sensing the changes in position of the guiding zone detect the angle of rotation of the guiding zone about the first and/or second axis, i.e. the degree of tilt of the guiding zone about the first or the second axis respectively. This type of detecting the position or changes in position of the guiding zone thus supplies as a result, data about the relative position or change in position relative to the neutral position of the guiding zone.

In another embodiment the position of the guiding zone may further be determined absolutely, i.e. without determining a rotation of the guiding zone about the first or the second axis, such as through optically recording the position of the guiding zone relative to a connecting point of the spacer with the sensor means or to any other point, such as through optoelectronically recording elements, such as photodiodes, phototransistors, or else CCD elements and the like. For example, the absolute position of the guiding zone may as well be determined by registering the position or the movement of the guiding zone in an electric, electromagnetic or magnetic field.

Preferably, recording of the angle of rotation of the guiding zone about the first and/or second axis is performed mechanically, electronically and/or optically.

To mechanically sense the angle of rotation of the guiding zone about the first or the second axis, a particularly preferred embodiment of the present invention comprises at least one element extending at least in part on a radius about the first and/or second axis, such as a guide and/or return pulley or else a lever element or the like. Such elements extending on a radius about the first or the second axis are connected with the guiding zone to be non-rotatable relative to tilting of the guiding zone about the respective axis, such that for example a rotation of the guiding zone about the first axis will result in a corresponding rotation of the element extending on a radius about the first axis, or a rotation of the guiding elements about the second axis, in a corresponding rotation of an element extending on a radius about the second axis.

Sensing the rotation of such elements extending at least in part on a radius about the first or second axis, in particular of pulleys or lever components, may also be performed mechanically or electronically or else optically, e.g. by means of optoelectronic sensors or with the aid of reflecting elements such as mirrors, or else through induction.

Most preferably the means for sensing changes in position of the guiding zone comprise at least one pull/push element, in particular a control cable element preferably configured as a Bowden cable and connected directly or indirectly with the guiding zone and/or an element extending at least in part radially about the first respectively second axis or else is in mechanical contact with it, for example in frictional contact.

The connection or the contact of the pull/push element or the control cable element with the guiding zone or an element radially extending about the first or second axis is such that tilting of the guiding zone about the first or second axis will result in a lengthening or shortening of the pull/push or control cable element, depending on whether the rotation angle or the tilt angle is positive or negative relative to the neutral position of the guiding element. A control cable element extending about a pulley or return element will be shortened as the guiding zone is tilted about the respective axis in a first section and in a second section, lengthened.

A particularly preferred embodiment of the present invention has the guiding zone radially distanced by a spacer element from the first and/or the second axis such that the distance between the guiding element and the first and/or second axis is preferably 1 to 20 cm, preferably 5 to 15 cm and most preferably approx. 10 cm.

Most preferably the spacer is configured such that the guiding zone is positioned relative to the first and/or second axis such that when the device is in use, i.e. while the user holds, grips or engages with the guiding zone, the wrist of that hand which guides the guiding zone, intersects with the first and/or second axis, or else it is in the immediate vicinity thereof. Most preferably the spacer is configured such that during use of the device the wrist of the user is at least near the first intersection point.

It is particularly preferred that the guiding zone comprises at least one actuating device which allows triggering or controlling a function of the instrument. Such actuating devices are for example, pressure- or pull-sensitive elements such as knobs, switches and the like, and forceps- or scissors-type, or finger-cot or glove-shaped elements where the instrument function can be controlled for example by varying the distance of two engaging elements, a tilting of the two elements relative to one another, by curving, pressing or pulling or the like, preferably continuously.

According to another particularly preferred embodiment the guiding zone comprises at least a plurality of actuating devices configured so as to be suitable to receive at least one finger each. The receiving means can receive one or more members of a finger in the shape of a “finger cot” with the actuating device in particular placed such that the movement of a received finger will result in movement of the tool.

Said finger cot in the sense of a closed sheath may receive the whole or a portion of the finger where according to a particularly preferred embodiment said finger cot is manufactured of metal or plastic and is adapted to the anatomy of the user's hand.

In a particularly preferred embodiment of said receiving means in particular the thumb and the index finger of a user's hand are each received in a “finger cot” where in particular in a so-called opposing movement i.e. where the two fingers move toward one another, preferably on one plane, the tool controlled via the device performs a corresponding movement such as e.g. an opening or closing movement in a corresponding plane.

Such a motion guidance may be called an “intuitive tool guidance”. Such “intuitive tool guidance” offers in particular the possibility that the user does not need to acquire movements different from those habitual in using scissors or clamps such that the familiar surgical techniques remain unchanged to the user. This is imperative in particular in cases of complicated sequences of motions where the user must be able to rely on familiar reactions of the tools used. Modifications of such tool guidance are as a matter of course included in the sense of the present invention.

Sensing the position or the condition of the actuating device preferably also occurs mechanically, electronically or optically and particularly preferably through a pull/push element connected with the actuating device, in particular a control cable element and preferably a Bowden cable.

In a particularly preferred embodiment of the present invention the connection of the sensor means with the spacer is configured releasable such that a sensor means can be used with a plurality of spacers and controllers or instruments. This offers the possibility to configure for example the spacer, the controller and the instrument attached thereto as sterile single-use items which can be thrown out after use while allowing a continued use of the sensor means with other instruments.

It is particularly preferred to select the type of connecting the spacer with the sensor means from a group including, form-fit, frictional and/or adhesive connections and in particular screwed connections, plug-in connections, snap-on connections, riveted connections, bayonet connections, crimped connections, clamping connections, glued connections, welded connections, integral connections and any combinations thereof and the like.

A preferred embodiment of the device according to the invention provides for transmission means which transmit to the controller data acquired about changes in position or the position of the guiding zone. Transmission occurs preferably electronically, through electromagnetic waves, or mechanically.

It is particularly preferred for the transmission means to comprise at least one pull/push element, in particular a control cable element by means of which data acquired by the sensor means about changes in position of the guiding zone relative to the neutral position is represented by a lengthened or shortened pull/push or control cable element at the controller. Preferably the pull/push or control cable elements of the transmission means are mechanically coupled and in particular integrally connected with the pull/push or control cable elements of the sensor means or the means for detecting the position of the guiding zone.

Most preferably the pull/push elements or control cable elements of the transmission means extend within the spacer and particularly preferably within the tubular hollow spaces or lumina limited by the spacer.

A particularly preferred embodiment of the present invention has the controller configured such that the acting direction of the instrument is variable at least in one plane and preferably at least in part in a substantially spherical, in particular a semispherical space segment. Herein the data acquired by the sensor means about the position of the guiding zone is transposed or transmitted indirectly or directly into a deflection of the controller correlating thereto.

Deflection of the controller is substantially carried out such that depending on the position of the guiding zone relative to the neutral position, the portion of the controller carrying the instrument points in a direction orientated substantially parallel to a perpendicular placed on a subrange of the spherical or semispherical space segment.

In one embodiment the means of the controller for changing the acting direction of the instrument comprise at least one electromechanical element, in particular a motor, a stepping motor or linear motor, a piezoelectric element and/or the like.

It is particularly preferred that the means of the controller for changing the acting direction of the instrument comprise a mechanical element, in particular a pull/push element, preferably a control cable element, a fixation and/or return element for a pull/push element or a control cable element, a spring element, a joint element, or the like. Preferably the pull/push element or control cable element of the controller are mechanically coupled and in particular integrally connected with the pull/push or control cable element of the sensor means respectively the pull/push or control cable elements of the transmission means.

It is particularly preferred that the joint element of the means for changing the acting direction is a flexible element, in particular an elastic element or a spring, extending at least in its neutral position at least in part on the axis of the spacer in the area of the second end where the controller is positioned. Such a joint element is preferably configured such that with the joint element being tilted, deflected or moved out of the neutral position, a restoring force acts on the joint element to return it to the neutral position.

In a particularly preferred embodiment of the present invention such a joint element connects the second end of the spacer with a fixation element at which the instrument is placed. Preferably the fixation element also has at least three pull/push elements, in particular control cable elements attached to it which in the neutral position of the joint element extend substantially parallel thereto and in the direction of the second end of the spacer, and which are coupled and in particular integrally connected with the pull/push elements or control cable elements of the transmission means or the sensor means.

The joint element is attached to the fixation element preferably substantially centrally and the pull/push elements substantially concentrically thereto. In relation to their number, the pull/push elements are attached to the fixation element substantially in a circle and substantially equidistantly relative to one another on the circle line.

In this configuration of the controller a shortening of any of the pull/push elements or control cable elements will result in a force component which is orthogonal relative to the neutral position of the joint element in the direction of the shortening pull/push element so as to thereby cause a deflection of the joint element out of its neutral position in the direction of the point where the shortening pull/push element is attached to the fixation means.

In another preferred embodiment the region of the second spacer end where the controller is placed has provided at it a limit stop which allows variations of the operating range of the controller or in other words which limits the amplitude or the angle of deflection of the controller out of the neutral position. Preferably the limit stop is displaceable at least over a portion of the spacer length which is a simple way of allowing variations in the operating range of the controller.

The device for controlling instruments according to the present invention is in particular suitable for controlling instruments or tools selected from a group including radiation instruments and recording instruments, in particular instruments for emitting or absorbing and for transmitting electromagnetic waves in the visible and near infrared or ultraviolet region and/or sound waves, and gripping and/or cutting instruments, mechanical, optical or electrical ablation instruments, sewing instruments and other instruments used in particular in the field of endoscopy, arthroscopy, thoracoscopy, laparoscopy, neurosurgery or cardiology, and the like.

Further features of the present invention follow below from the detailed description of a specific embodiment in conjunction with the figures and the claims.

There are shown in:

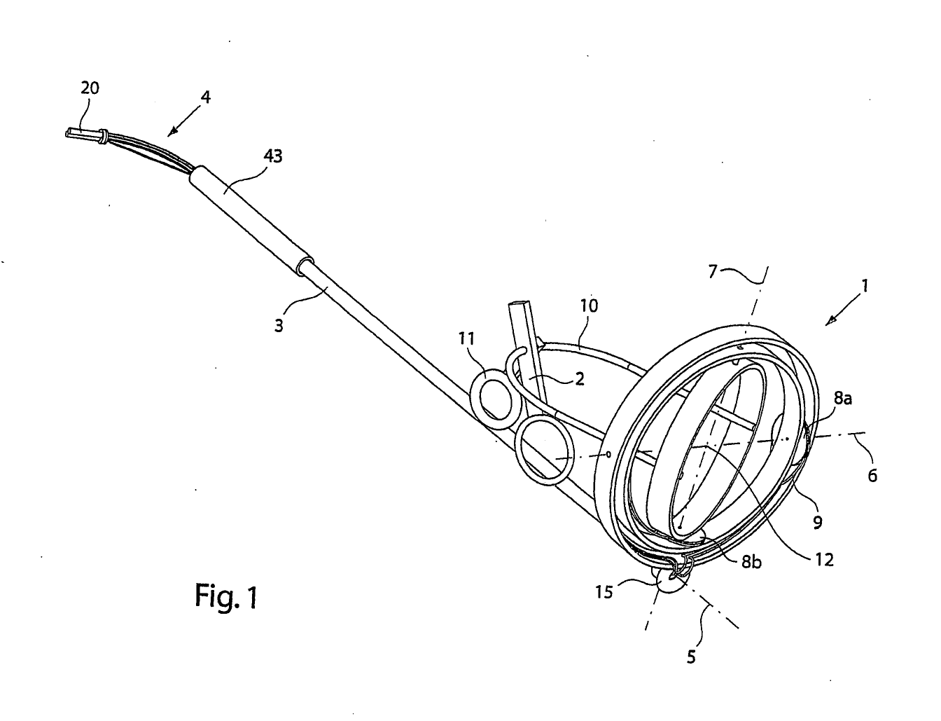

FIG. 1 a device for controlling instruments according to the invention;

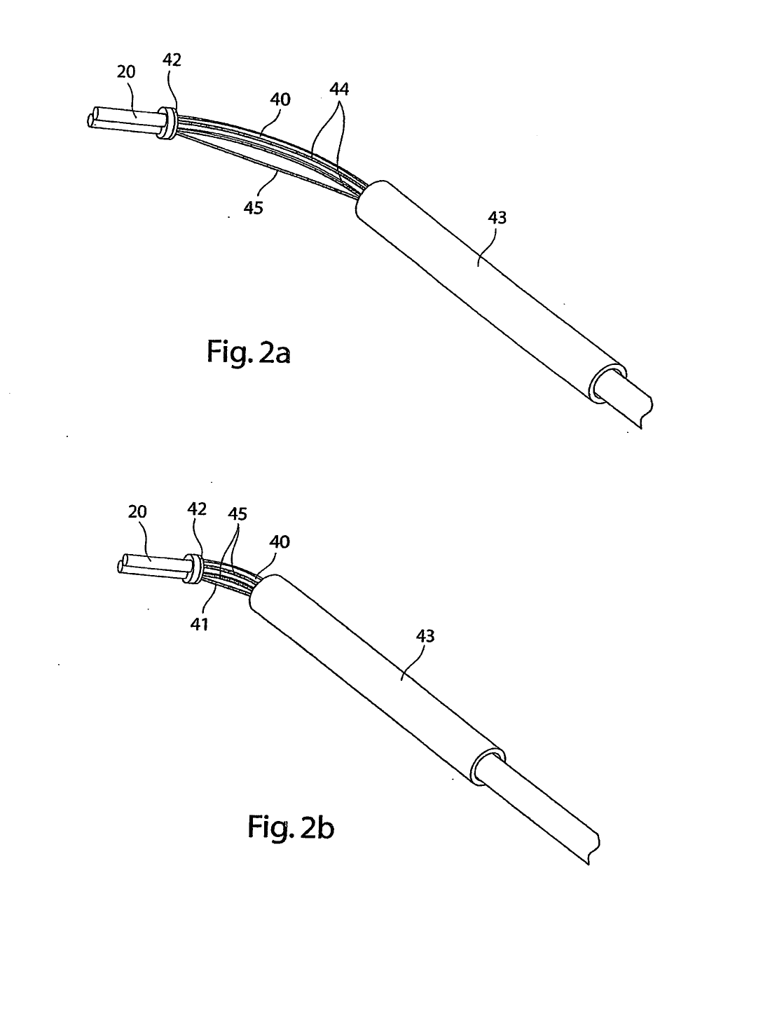

FIG. 2a a controller of a device according to the invention as in FIG. 1;

FIG. 2b another view of a controller of a device according to the invention as in FIG. 1.

FIG. 1 shows a device for controlling instruments according to the invention comprising a sensor means 1, a spacer 3, and a controller 4. The spacer 3 consists of an elongated, rigid and substantially tubular element and is connected with the sensor means 1 in the region of a first end. In the region of its second end, the spacer is connected with the controller 4 which carries an instrument 20 to be controlled.

The sensor means 1 comprises a guiding zone 2 which is supported to be rotatable about a first axis 6 which, in the region of the connection 15 with the sensor means, is substantially orthogonal to the axis 5 of the principal dimensional direction of the spacer. Moreover the guiding zone 2 is supported to be rotatable about a second axis 7 which is substantially orthogonal relative to the first axis 6 wherein said first axis 6 and said second axis 7 intersect in a first intersection point 12. The first axis 6 and the second axis 7 are furthermore placed substantially orthogonally to the axis 5 of the principal dimensional direction of the spacer in the region of the connection 15 with the sensor means.

The rotatable support of the guiding zone 2 about the first axis 6 or the second axis 7 forms a cardan joint by means of which the guiding zone 2 is configured as a gimbal suspension relative to an axis which is substantially parallel to the axis 5 of the principal dimensional direction of the spacer.

The connection of the sensor means with the spacer of the device according to the invention is rigid such that the position of the plane opened by said first axis 6 and said second axis 7 does not vary relative to the axis 5 of the principal dimensional direction of the spacer.

The guiding zone 2 is radially spaced apart from the first axis 6 or the second axis 7, respectively, and in particular from their first intersection point 12, wherein the distance of the guiding zone from the first intersection point 12 is approx. 10 cm which results in that the wrist of the user, who during use for example engages his thumb in the guiding zone 2 for guiding it, is positioned approximately in the region of the first intersection point 12. The guiding zone 2 furthermore comprises an actuating device 11 which in conjunction with the guiding zone 2 is configured like scissors or finger cots and which allows to control the function of the instrument 20 in the way scissors are used, meaning that the distance between the thumb engaging with the guiding zone 2 and another finger of the user's hand engaging with the actuating device 11, controls the function of the instrument preferably continuously.

The sensor means of the device according to the invention in FIG. 1 furthermore comprises an element 8a in the form of a return pulley radially extending about the first axis 6 and an element 8b in the form of a return pulley radially extending about the second axis 7. The elements 8a, 8b radially extending about the first or the second axis are connected via the spacer 10 with the guiding zone 2 to be non-rotatable relative to a rotation about the first axis 6 or the second axis 7.

The sensor means of the device according to the invention in FIG. 1 furthermore comprises pull/push elements, in particular control cable elements and preferably Bowden cables 9 which run around the return pulleys 8a, 8b, thus interacting with these such that a tilting of the guiding zone about the first axis 6 or the second axis 7 will result in that portions of the Bowden cable will lengthen or shorten respectively before or after circling the pulley. The detection of the angle of rotation or tilting of the guiding zone 2 about the first axis 6 or the second axis 7 thus occurs mechanically by way of a transmission of the tilt angle into a lengthening or shortening of the portions of the control cable element or the Bowden cable 9.

The device according to the invention in FIG. 1 furthermore comprises transmission means in the form of control cables which are connected with the control cables 9 in the region of the connection 15, in particular integrally connected, and which represent the lengthening or shortening of the portions of the control cable elements or Bowden cables 9 by way of a lengthening or shortening at the controller 4. The controller of the device according to the invention in FIG. 1 employs a transmission of the data about the position of the guiding zone relative to the neutral position represented by way of a lengthening or shortening to enable changes in the acting direction of the instrument 20 preferably in a substantially spherical space segment. The direction of the instrument 20 correlates with the position of the guiding zone 2 in that a rotation of the guiding zone about the second axis 7, which leads to a tilting movement of the guiding zone 2 in a horizontal plane, likewise leads to a change in direction of the instrument 20 in a horizontal plane and in particular in the direction of tilt of the guiding zone out of the neutral position and likewise a tilting of the guiding zone 2 about the first axis 6, which leads to a movement of the guiding zone in a vertical plane, results in a change in the acting direction of the instrument 20 through the controller 4 in a vertical plane.

The FIGS. 2a and 2b are enlarged illustrations of the controller 4 of the device according to the invention in FIG. 1. The instrument 20 is placed at a fixation element 42 of the controller, which in turn is placed on the side of the controller facing away from the sensor means and which is spaced apart from the second end of the spacer 3 by a joint element 40, which is in particular configured in the shape of an elongated, flexible element, in particular a metal spring or a plastic spring. When the joint element is deflected out of its neutral position which substantially corresponds to a linear extension, said joint element 40 has, in particular in the region of the fixation element, a restoring force with a component in the direction of the neutral position.

The controller according to the FIGS. 2a and 2b furthermore comprises four control cable elements, preferably Bowden cables 44, 45 which correspond with the portions of the control cable elements 9 circling the return rolls 8a respectively 8b and which are in particular configured integrally. The control cable elements 44, 45 are connected with the fixation means 42 such that they are arranged concentrically relative to the connecting point of the fixation means 42 with the joint element 40 wherein the control cable elements are connected to the fixation means 42 at connecting points located and substantially equidistantly placed, i.e. spaced apart at substantially regular intervals, on a circular path that is substantially concentric relative to the fixation point of the joint element 40.

As is shown in the FIGS. 2a and 2b, a shortening of a control cable element 45 will, due to a corresponding change in position of the guiding zone 2 relative to the neutral position result in a corresponding change in position of the acting direction of the instrument 20, which in respect of the direction and the amplitude of the deflection substantially correlates with the change in position of the guiding zone relative to the neutral position. In the case of the FIGS. 2a and 2b a shortening of one of the four control cables connected with the fixation means 42 will result in a change of the acting direction of the instrument 20 on a horizontal plane.

The FIGS. 2a and 2b also show a limit stop 43 which can be displaced on the spacer 3 such that the reference point of the controller for deflecting or changing the direction of the instrument is thus displaceable such that the operating range of the controller can be varied or the direction and/or amplitude of deflection of the controller out of the neutral position can be limited.

A displacement of the limit stop 43 on the spacer 3 in the direction of the sensor means will thus lead to a lengthening of the effective length of the joint element of the controller, such that in particular the amplitude of deflection of the controller and thus the operating range of the controller or the instrument placed thereat expands.

In contrast to this, the limit stop in FIG. 2b is displaced in the direction of the side of the controller facing the instrument, such that the effective length of the joint element shortens correspondingly and thus the amplitude of deflection or the operating range of the controller is reduced.

The device according to the invention provides for a transmission of the restoring force acting in the direction of the neutral position of the controller through the control cable elements or pull/push elements toward the sensor means so as to guarantee an automatic transfer of both the controller and the sensor means to their respective neutral positions.

1. A device for controlling an instrument comprising:
at least one sensor means comprising at least one guiding zone which is guided by a user's hand during use;
at least one spacer whose first end is rigidly fixed to the sensor means;
at least one controller which is placed at a second end of the spacer and carries at least one instrument to be controlled;
the sensor means comprises means for detecting changes in position of the guiding zoned out of a neutral position; and
the controller comprises means for controlling the acting direction of the instrument based on data acquired by the sensor means.

2. The device according to claim 1, characterised in that the guiding zone is supported to be rotatable about a first axis which, in the region of the connection with the sensor means, is substantially orthogonal to the axis of the principal dimensional direction of the spacer.

3. The device according to claim 2, characterised in that the guiding zone is supported to be rotatable about a second axis which is substantially orthogonal to the first axis.

4. The device according to claim 1, characterised in that the first and the second axis intersect one another.

5. The device according to claim 2, characterised in that the means for detecting a change in position of the guiding zone detect the angle of rotation of the guiding zone about the first and/or second axis.

6. The device according to claim 5, characterised in that detection of the angle of rotation is performed mechanically, electronically and/or optically.

7. The device according to claim 2, characterised in that the means for detecting the change in position of the guiding zone comprise at least one element which extends at least in part on a radius about the first and/or second axis and which is rigidly connected with it relative to the movement of the guiding zone about the first or the second axis.

8. The device according to claim 2, characterised in that the means for detecting the change in position of the guiding zone comprise at least one pull/push element, in particular a control cable element which is in direct or indirect contact with the guiding zone and/or with the element which radially extends about the first and/or the second axis, such that a change in position of the guiding zone about the first or the second axis will result in a lengthening or shortening of the pull/push element or the control cable element.

9. The device according to claim 2, characterised in that the guiding zone is radially spaced apart from the first axis and/or the second axis by a spacer wherein each distance is 1 cm to 20 cm, preferably 5 cm to 15 cm and particularly preferred approximately 10 cm.

10. The device according to claim 1, characterised in that the guiding zone comprises at least one actuating device by means of which a function of an instrument can be triggered or controlled.

11. The device according to claim 1, characterised in that the connection of the sensor means with the spacer is releasable.

12. The device according to claim 1, characterised in that transmission means are provided which transmit to the controller data acquired by the sensor about changes in position of the guiding zone.

13. The device according to claim 12, characterised in that the transmission means transmit data about the position of the guiding zone electronically, through electromagnetic waves, or mechanically.

14. The device according to claim 12, characterised in that the transmission means comprise at least one pull/push element, in particular a control cable element by means of which data acquired by the sensor means about changes in position of the guiding zone relative to the neutral position is represented at the controller by a lengthened or shortened pull/push element or control cable element.

15. The device according to claim 1, characterised in that the acting direction of the instrument is variable at least in one plane and preferably in a substantially spherical, in particular in a semispherical space segment, by means of the controller.

16. The device according to claim 1, characterised in that the control means for changing the acting direction of the instrument comprise at least one electromechanical element, in particular a motor, preferably a stepping motor or linear motor, or a piezoelectric element.

17. The device according to claim 1, characterised in that the control means for changing the acting direction of the instrument comprise at least one mechanical element, in particular a pull/push element, preferably a control cable element and most preferably a Bowden cable, a fixation and/or return element for a pull/push element or a control cable element, a spring element, or a joint element.

18. The device according to claim 1, characterised in that the control means for changing the acting direction of the instrument comprise at least one flexible element, preferably a flexible element exhibiting a restoring force acting toward the neutral position of the instrument, and at least three pull/push elements, in particular control cable elements attached to a fixation element on the side of the controller facing toward the instrument such that a shortening of at least one pull/push element or control cable element will result in a change of the acting direction of the instrument.

19. The device according to claim 1, characterised in that the controller comprises at least one limit stop by means of which the operating range of the controller can be varied.

20. The device according to claim 1, characterised in that the instrument is selected from a group of instruments including radiation instruments and recording instruments, in particular instruments for emitting, absorbing or transmitting electromagnetic waves in the visible and near infrared or ultraviolet region and/or sound waves, and gripping and/or cutting instruments, mechanical, optical or electrical ablation instruments, sewing instruments and other instruments used in particular in the field of endoscopy, arthroscopy, thoracoscopy, laparoscopy, neurosurgery or cardiology.
